(19) United States
(12) Patent Application Publication
(10) Pub. No.: US 2024/0225916 A1
(43) Pub. Date: Jul. 11, 2024

(54) ABSORBENT ARTICLE

(71) Applicant: Essity Hygiene and Health Aktiebolag, Göteborg (SE)

(72) Inventors: Philip BLOMSTRÖM, Göteborg (SE); Lisa PALMQVIST, Göteborg (SE); Anna KNÖS, Göteborg (SE)

(73) Assignee: Essity Hygiene and Health Aktiebolag, Göteborg (SE)

(21) Appl. No.: 18/560,297

(22) PCT Filed: May 7, 2021
(86) PCT No.: PCT/EP2021/062165

§ 371 Date: Nov. 10, 2023

(51) Int. Cl. A61F 13/49; A61F 13/15; A61F 13/513; A61F 13/514; A61F 13/534

(52) U.S. Cl. CPC A61F 13/49006; A61F 13/15268; A61F 13/513; A61F 13/51456; A61F 2013/15276; A61F 2013/15365; A61F 2013/15406; A61F 2013/49074; A61F 2013/53445

(57) ABSTRACT

The disclosure provides an absorbent undergarment comprising an integrated absorbent assembly, having an extension in the longitudinal and transverse directions. The assembly comprises an absorbent core comprising first and second absorbent layers, and having front, crotch and rear portions, and front and rear transverse edges. The first and second absorbent layers overlap in the crotch portion. The first absorbent layer has a first layer frontmost half and a rearmost half, the first absorbent layer having front and rear transverse edges. The second absorbent layer has a rearmost half and frontmost half, the second absorbent layer has a rear transverse edge and front transverse edge, wherein a maximum width of the first layer frontmost half is greater than that of the first layer rearmost half and wherein a maximum width of the second layer rearmost half is greater than that of the second layer frontmost half.

### TECHNICAL FIELD

The present disclosure pertains to a washable and reusable absorbent undergarment and a washable and reusable absorbent pad comprising an absorbent assembly. In particular, this disclosure pertains to a washable and reusable absorbent undergarment, and a washable and reusable absorbent pad, comprising an absorbent assembly including an absorbent core comprising a first and a second absorbent layer.

### BACKGROUND OF THE INVENTION

The present disclosure generally relates to the field of reusable and washable absorbent undergarments, and more particularly to a reusable and washable absorbent undergarment comprising an integrated absorbent assembly.

An alternative to disposable sanitary napkins and disposable absorbent undergarments are washable and reusable absorbent undergarment and washable and reusable absorbent pads. This may be an environmentally and economically advantageous replacement for disposable sanitary articles and disposable absorbent undergarments.

Washable and reusable absorbent undergarments conventionally comprise absorbent cores conventionally including woven, knit or cloth material. Such core materials have lower absorbent capacity compared to materials used for conventional absorbent cores in disposable absorbent articles, such as superabsorbent polymers and cellulosic fibers. The risk of leakage and rewetting may therefore be higher for reusable and washable absorbent undergarments. To increase the absorbency and thereby reducing the risk of leakage, such absorbent cores may comprise multiple core layers. However, such constructions may form a bulky structure being less comfortable compared to thinner disposable absorbent core including superabsorbent polymers and/or cellulosic fibers.

There is thus still a need for a reusable and washable absorbent undergarment which has improved leakage security and comfort, and which is less bulky.

### SUMMARY OF THE INVENTION

One or more of the above objects may be achieved with a washable and reusable outer garment in accordance with claim 1 and a washable and reusable pad according to claim 20. Further embodiments are set out in the dependent claims and in the following description.

The present disclosure relates to a washable and reusable absorbent undergarment comprising an integrated absorbent assembly. The absorbent undergarment has an extension in the longitudinal direction and in the transverse direction. The absorbent assembly comprises an absorbent core, the absorbent core comprising a first absorbent layer and a second absorbent layer. The absorbent core has a front portion, a crotch portion and a rear portion. The absorbent core furthermore has a core front transverse edge and a core rear transverse edge. The first absorbent layer and the second absorbent layer overlap each other in the crotch portion of the absorbent core. The first absorbent layer has a first layer frontmost half and a first layer rearmost half. The first absorbent layer has a first layer front transverse edge, and a first layer rear transverse edge, the second absorbent layer has a second layer rearmost half and a second layer frontmost half. The second absorbent layer has a second layer rear transverse edge, and a second layer front transverse edge, wherein a maximum width of the first layer frontmost half is greater than a maximum width of the first layer rearmost half and wherein a maximum width of the second layer rearmost half is greater than a maximum width of the second layer frontmost half.

The washable and reusable absorbent undergarment is an absorbent undergarment that is capable of being washed without essential shrinkage or other damage, such as after five and up to forty washing occasions. The absorbent undergarment is made from a washable material such as a washable fabric. The absorbent assembly may comprise woven material, a nonwoven and/or a knit material. The fibers in the absorbent assembly material may be synthetic fiber, such as polyamid, polyester and polyolefin fibers, and/or wool fibers, cotton fibers and/or rayon fibers.

The first absorbent layer may an upper core layer and the second absorbent layer may be a lower core layer. Alternatively, the second absorbent layer may be the upper core layer and the first absorbent layer may be the lower core layer. The absorbent assembly may comprise a body-facing layer and the upper core layer may then be arranged between the body-facing layer and the lower core layer.

A minimum width of the first layer rearmost fourth may be ±80% of a width of a first layer crotch part, the width of the first layer crotch part being measured at a smallest width of the absorbent core where the first absorbent layer and the second absorbent layer overlap each other in the crotch portion.

A minimum width of the second layer frontmost fourth may be ±80% of a width of the second layer crotch part, the width of the second layer crotch part being measured at a smallest width of the absorbent core where the first absorbent layer and the second absorbent layer overlap each other in the crotch portion.

The front portion may constitute of 30% of the absorbent core, as seen in the longitudinal direction L and from a core front edge of the absorbent core. The crotch portion may constitute of 40% of the absorbent core and the rear portion may constitute of 30% of the absorbent core, as seen in the longitudinal direction.

The first absorbent layer extends at least in the front portion and in the crotch portion and the second absorbent layer extends at least in the rear portion and in the crotch portion.

The first absorbent layer frontmost edge may correspond to the frontmost edge of the absorbent core.

The second absorbent layer rearmost edge may correspond to the rearmost edge of the absorbent core.

The materials used in absorbent core in washable and reusable absorbent undergarments have lower absorbent capacity than convention absorbent core materials in disposable absorbent article. To compensate for the lower absorbency, additional material and/or material layers may be included into the absorbent core of the washable and reusable absorbent undergarment. This may however lead to bulkier and less comfortable and discreet absorbent undergarments.

In a washable and reusable absorbent undergarment comprising an integrated absorbent assembly both problems are solved by providing the absorbent assembly with a first and second absorbent layer and by arranging the layers to overlap in the crotch region and by providing the first absorbent layer with a first layer frontmost half having a maximum width that is greater than the maximum width of the first layer rearmost half and by providing the second layer with a second layer rearmost half with a maximum width that is greater than a maximum width second layer frontmost half. Consequently, the crotch region of the absorbent assembly has an enhanced absorbency while the front and rear sections of the absorbent assembly are less bulky and thus both more discreet and comfortable.

The maximum width of the first layer frontmost half may be at least 5% greater than the maximum width of the first layer rearmost half. Optionally, the maximum width of the first layer frontmost half may be at least 10%, or 15%, greater than the maximum width of the first layer rearmost half. Optionally, the maximum width of the first layer frontmost half may be at least 20%, 30% or 40% greater than the maximum width of the first layer rearmost half. The maximum width of the first layer frontmost half may be up to 350%, such as up to 200%, greater than the maximum width of the first layer rearmost half.

The maximum width of the second layer rearmost half may be at least 5% greater than the maximum width of the second layer frontmost half. Optionally, the maximum width of the second layer rearmost half may be at least 10%, or 15%, greater than the maximum width of the second layer frontmost half. Optionally, the maximum width of the second layer rearmost half may be at least 20%, 30% or 40% greater than the maximum width of the second layer frontmost half. The maximum width of the second layer rearmost half may be up to 350%, such as up to 200%, greater than the maximum width of the second layer frontmost half.

The maximum width of the first layer rearmost half may be within the range of from −80% to ±20% of a width of a first layer crotch part, the width of the first layer crotch part being measured at a smallest width of the absorbent core where the first absorbent layer and the second absorbent layer overlap each other in the crotch portion.

The first layer rearmost half, such as a rear end portion of the first layer, may thus have a width which is the same or only slightly broader or narrower at a rearmost end of the first layer than at a crotch section first layer. This provides a rear portion of the absorbent assembly with a higher absorbency in a central part, as seen in the transverse direction, while the lateral parts of the rear portion only have one absorbent layer, thus providing an absorbent undergarment which is less bulky and more comfortable and discreet to wear.

The maximum width of the second layer frontmost half may be within the range of from −80% to +20% of a width of the first layer crotch part, the width of the second layer crotch part being measured at a smallest width of the absorbent core where the first absorbent layer and the second absorbent layer overlap each other in the crotch portion.

The second layer frontmost half, such as a front end portion of the second layer, may thus have a width which is the same or only slightly broader or narrower at a frontmost end of the second layer than at a crotch section of the second layer. This provides a rear portion of the absorbent assembly with a higher absorbency in a central part, as seen in the transverse direction, while the lateral parts of the rear portion only have one absorbent layer, thus providing an absorbent undergarment being less bulky and more comfortable and discreet to wear.

A width of the first absorbent layer may differ from a width of the second absorbent layer along an area of the absorbent core where the first absorbent layer and the second absorbent layer overlap each other in the crotch portion. Optionally, a width of the first absorbent layer may differ from a width of the second absorbent layer along the entire area of the absorbent core where the first absorbent layer and the second absorbent layer overlap each other in the crotch portion. This provides a less bulky and distinct edge region in the crotch portion.

The first absorbent layer may have a length, as measured along a longitudinal centerline of the absorbent undergarment, corresponding to from 35% to 100%, such as from 35% to 95%, or from 35% to 85%, of a total length of the absorbent core, between the front transverse edge of the absorbent core and the rear transverse edge of the absorbent core. The first absorbent layer does thus not cover the total length of the absorbent core and the rear portion of the absorbent assembly is thinner than a crotch portion of the absorbent assembly.

The first absorbent layer may have a length, as measured along a longitudinal centerline of the absorbent undergarment, corresponding to from 20% to 100%, such as from 25% to 100% or from 30% to 100%, of a total length of the absorbent core, between the front transverse edge of the absorbent core and the rear transverse edge of the absorbent core. In an alternative embodiment the first absorbent layer may have a length, as measured along a longitudinal centerline of the absorbent undergarment, corresponding to from 15% to 40%, of a total length of the absorbent core, between the front transverse edge of the absorbent core and the rear transverse edge of the absorbent core. The second absorbent layer may have a length, as measured along a longitudinal centerline of the absorbent undergarment, corresponding to from 35% to 100%, such as from 35% to 95%, or 35% to 85%, of a total length of the absorbent core, between the front transverse edge of the absorbent core and the rear transverse edge of the absorbent core. The second absorbent layer does thus not cover the total length of the absorbent core and the front portion of the absorbent assembly is thinner than a crotch portion of the absorbent assembly.

The second absorbent layer may have a length, as measured along a longitudinal centerline of the absorbent undergarment, corresponding to from 20% to 100%, such as from 25% to 100% or from 30% to 100%, of a total length of the absorbent core, between the front transverse edge of the absorbent core and the rear transverse edge of the absorbent core. In an alternative embodiment the second absorbent layer may have a length, as measured along a longitudinal centerline of the absorbent undergarment, corresponding to from 15% to 40%, of a total length of the absorbent core, between the front transverse edge of the absorbent core and the rear transverse edge of the absorbent core.

Optionally, at least a section of the second layer rear transverse edge has a radius of curvature and wherein at least a section of a first layer rear transverse edge has a radius of curvature of from ±70% of the radius of curvature of the at least a section of the second layer rear transverse edge. The section of the second layer rear transverse edge may have a length corresponding to from 20% to 100%, such as from 40% to 100%, of a total length of the second layer rear transverse edge and the section of the first layer rear transverse edge may have a length corresponding to from 20% to 100%, such as from 40% to 100%, of a total length of the first layer rear transverse edge. Optionally, the entire second layer rear transverse edge has a radius of curvature and wherein at the entire first layer rear transverse edge has a radius of curvature of from ±70% of the radius of curvature of the second layer rear transverse edge.

Since the absorbent garment is a washable and reusable absorbent garment the absorbent assembly may be attached by sewing. For an absorbent core according to the present disclosure wherein a section of the second layer rear transverse edge has a radius of curvature and wherein at least a section of a first layer rear transverse edge has a radius of curvature of from ±70% of the radius of curvature of the at least a section of the second layer rear transverse edge provides an even and smoother surface in the rear portion of the absorbent core.

Optionally, at least a section of the first layer front transverse edge has a radius of curvature and wherein at least a section of a second layer front transverse edge has a radius of curvature of from ±70% of the radius of curvature of the at least a section of the first layer front transverse edge. The section of the first layer front transverse edge may have a length corresponding to from 20% to 100%, such as from 40% to 100%, of a total length of the first layer front transverse edge and the section of the second layer front transverse edge may have a length corresponding to from 20% to 100%, such as from 40% to 100%, of a total length of the second layer front transverse edge. Optionally, the entire first layer front transverse edge has a radius of curvature and wherein the entire second layer front transverse edge has a radius of curvature of from ±70% of the radius of curvature of the first layer front transverse edge.

For an absorbent core according to the present disclosure wherein a section of the first layer front transverse edge has a radius of curvature and wherein at least a section of a second layer front transverse edge has a radius of curvature of from ±70% of the radius of curvature of the at least a section of the first layer front transverse edge provides an even and smoother surface at the front portion of the absorbent core.

The absorbent core may comprise a third absorbent layer, the third absorbent layer extending over the crotch portion of the absorbent core.

The third absorbent layer may have a width being either smaller or greater than a respective width of the first and second absorbent layer in a respective first and second layer crotch part. This may reduce a bulky and distinct edge region in the crotch portion. The third absorbent layer may have a width being either smaller or greater than a respective width of the first and second absorbent layer along the entire length of the third absorbent layer.

The first absorbent layer and the second absorbent layer may together form a dog-bone shaped absorbent core.

The absorbent assembly may comprise a body-facing layer having a basis weight of from 100 gsm. The absorbent assembly may comprise a body-facing layer having a basis weight of from 120 gsm. To have a thicker body-facing layer over the first and second layer which have outer edges not coinciding with each other may mask the edge region of the upper layer of the first and second layer and thereby provide a more comfortable body-facing surface.

The absorbent assembly may comprise one or more of the following: a woven material, a nonwoven and/or a knit material.

The absorbent assembly may comprise a backsheet barrier layer. The backsheet barrier layer may comprise a plastic film.

According to a further aspect, the present disclosure relates to an absorbent pad comprising an absorbent assembly comprising a body-facing layer, a backsheet barrier layer and an absorbent core interposed between the body-facing layer and the backsheet barrier layer. The absorbent core comprises a first absorbent layer and a second absorbent layer, the absorbent core having a front portion, a crotch portion and a rear portion. The absorbent core has a core front transverse edge and a core rear transverse edge. The first absorbent layer and the second absorbent layer overlap each other in the crotch portion of the absorbent core. The first absorbent layer has a first layer frontmost half and a first layer rearmost half, the first absorbent layer having a first layer front transverse edge, and a first layer rear transverse edge. The second absorbent layer has a second layer rearmost half and a second layer frontmost half, the second absorbent layer having a second layer rear transverse edge, and a second layer front transverse edge. A maximum width of the first layer frontmost half is greater than a maximum width of the first layer rearmost half and wherein a maximum width of the second layer rearmost half is greater than a maximum width of the second layer frontmost half.

The absorbent assembly of the absorbent pad may be an absorbent assembly according to the preceding disclosure.

### BRIEF DESCRIPTION OF THE DRAWINGS

The present invention will be further explained hereinafter by means of non-limiting examples and with reference to the appended drawings wherein:

FIG. 1 illustrates a washable and reusable absorbent undergarment according to the present disclosure,

FIG. 2 illustrates the washable and reusable absorbent undergarment shown in FIG. 1 in a plan view; and

FIG. 3 is a plan view of a washable and reusable absorbent undergarment according to the present disclosure.

### DETAILED DESCRIPTION

The invention will be described more closely below by reference to an exemplary embodiment. The invention may however be embodied in many different forms and should not be construed as limited to the embodiments set forth in the drawings and the description thereto.

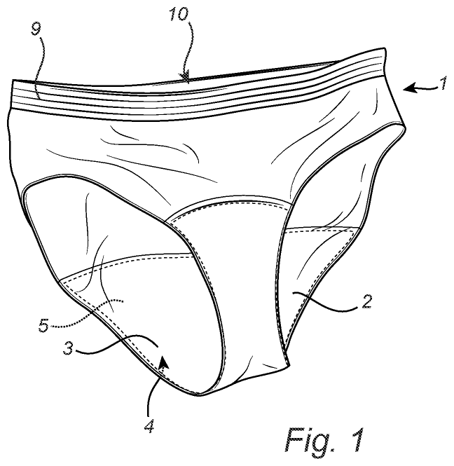

FIG. 1 illustrates a washable and reusable undergarment 1 according to the present disclosure. The absorbent undergarment 1 comprises an integrated absorbent assembly 2. The integrated absorbent assembly 2 comprises a body-facing layer 3, a backsheet barrier layer 4 and an absorbent core 5 interposed between the body-facing layer 3 and the backsheet barrier layer 4. The integrated absorbent assembly 2 is attached to a main material, such as a main fabric, of the washable and reusable undergarment 1. The main material may be a conventional undergarment material, for example of a convention panty material. The washable and reusable undergarment 1 is provided with waist elastics 9 along a waist opening 10 of the washable and reusable absorbent undergarment 1.

The washable and reusable absorbent undergarment 1 is an absorbent undergarment 1 that is capable of being washed without essential shrinkage or other damage, such as after five and up to forty washing occasions. The absorbent undergarment 1 is made from a washable material such as a washable fabric. The integrated absorbent assembly 2 may comprise woven material, a nonwoven and/or a knit material. The fibers in the absorbent assembly material may be synthetic fiber, such as polyamid, polyester and polyolefin fibers, and/or wool fibers, and the other one of the first or the second absorbent layer comprises cotton fibers and/or rayon fibers. The backsheet barrier layer may comprise a plastic film, the plastic film may be a breathable plastic film. The body-facing layer may have a basis weight of from 100 gsm or from 120 gsm. The body-facing layer may have a basis weight of up to 250 gsm. The body-facing layer may be a woven material, a nonwoven and/or a knit material layer.

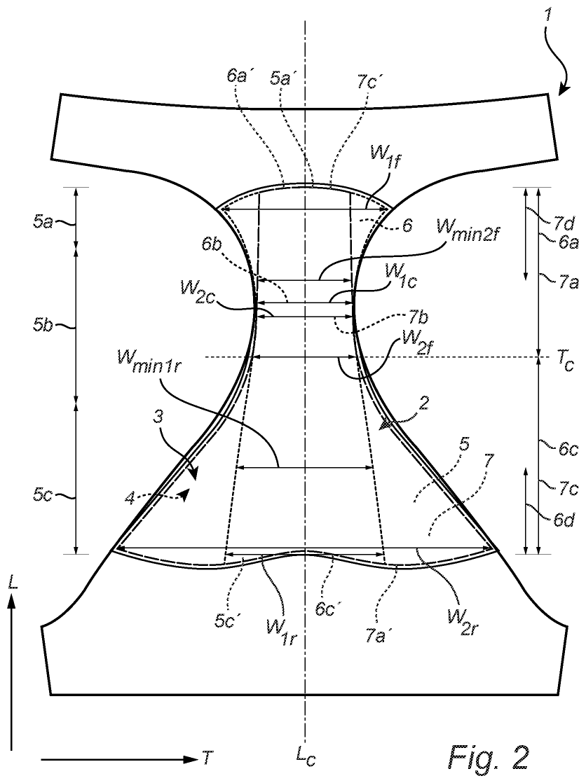

FIG. 2 illustrates the washable and reusable undergarment 1 from FIG. 1 in a plan view and as seen from a user facing side.

The absorbent undergarment 1 has an extension in the longitudinal direction L and in the transverse direction T. The absorbent assembly 2 comprises a body-facing layer 3 and a backsheet 4 and an absorbent core 5 interposed between the body-facing layer 3 and the backsheet 4. The absorbent core 5 comprises a first absorbent layer 6 and a second absorbent layer 7. The absorbent core 5 has a front portion 5a, a crotch portion 5b and a rear portion 5c. The absorbent core 5 has a core front transverse edge 5a′ and a core rear transverse edge 5c′. The first absorbent layer 6 and the second absorbent layer 7 overlap each other in the crotch portion 5b of the absorbent core 5 thus providing an improved absorbency in the crotch portion 5b of the absorbent core 5.

The first absorbent layer 6 has a first layer frontmost half 6a and a first layer rearmost half 6c, the first absorbent layer 6 furthermore has a first layer front transverse edge 6a′, and a first layer rear transverse edge 6c′. The second absorbent layer 7 has a second layer rearmost half 7c and a second layer frontmost half 7a, the second absorbent layer 7 furthermore has a second layer rear transverse edge 7c′, and a second layer front transverse edge 7a′. The first layer front transverse edge 6a′ corresponds to the core front transverse edge 5a′ and the second layer rear transverse edge 7c′ corresponds to the core rear transverse edge 5c′.

Each of the first absorbent layer 6 and the second absorbent layer 7 are asymmetrical as seen over a transverse centerline T_{c} of the absorbent assembly 2. A maximum width W_{1f} of the first layer frontmost half 6a is greater than a maximum width W₁ᵣ of the first layer rearmost half 6c and a maximum width W₂ᵣ of the second layer rearmost half 7c is greater than a maximum width W_{2f} of the second layer frontmost half 7a. The maximum width W_{1f} of the first layer frontmost half 6a may be at least 15% greater, or at least 20% greater than the maximum width W₁ᵣ of the first layer rearmost half 6c. The maximum width W_{1f} of the first layer frontmost half 6a may be from 15% to 350% greater, or from 20% to 200% greater, than the maximum width W₁ᵣ of the first layer rearmost half 6c. The maximum width W₂ᵣ of the second layer rearmost half 7c may be from 15% to 350% greater, or from 20% to 200% greater, than the maximum width W_{2f} of the second layer frontmost half 7a.

As may be seen in FIG. 2, the maximum width W₁ᵣ of the first layer rearmost half 6c is within the range of from −80% to ±20% of a width w_{1c} of a first layer crotch part 6b, the width w_{1c} of a first layer crotch part 6b being measured at a smallest width of the absorbent core 5 where the first absorbent layer 6 and the second absorbent layer 7 overlap each other in the crotch portion 5b. The maximum width W_{2f} of the second layer frontmost half 7a is within the range of from −80% to ±20% of a width w_{2c} of the second layer crotch part 7b, the width w_{2c} of a second layer crotch part 7b being measured at a smallest width of the absorbent core 5 where the first absorbent layer 6 and the second absorbent layer 7 overlap each other in the crotch portion 5b.

A minimum width Wₘᵢₙ₁ᵣ of the first layer rearmost fourth 6d may be ±80% of the width w_{1c} of a first layer crotch part 6b. The minimum width Wₘᵢₙ₁ᵣ of the first layer rearmost fourth 6d may be within the range of from −80% to ±20% of the width w_{1c} of a first layer crotch part 6b. The minimum width being measured in the transverse direction T of the absorbent undergarment 1.

A minimum width W_{min2f} of the second layer frontmost fourth 7d may be ±80% of a width w_{2c} of the second layer crotch part 7b. The minimum width W_{min2f} of the second layer frontmost fourth 7d may be within the range of from −80% to ±20% of a width w_{2c} of the second layer crotch part 7b.

The minimum width being measured in the transverse direction T of the absorbent undergarment 1.

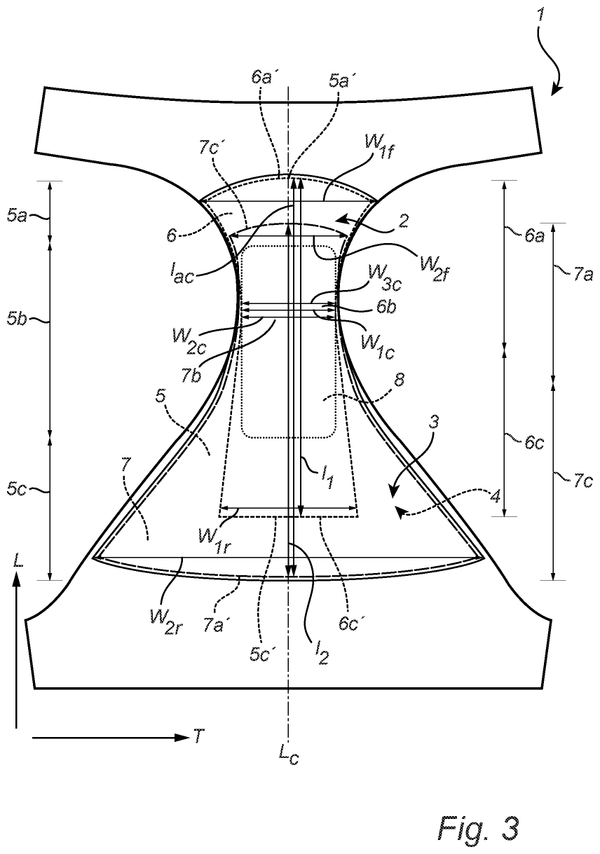

FIG. 3 illustrates a washable and reusable absorbent undergarment 1 according to the present disclosure. The same reference signs are used in FIG. 3 as in FIGS. 1 and 2 to denote the same or similar features.

The absorbent undergarment 1 has an extension in the longitudinal direction L and in the transverse direction T. The absorbent assembly 2 comprises a body-facing layer 3, a backsheet 4 and an absorbent core 5 arranged between the body-facing layer 3 and the backsheet 4. The absorbent assembly 2 may be attached to the undergarment by for example sewing. The absorbent core 5 comprises a first absorbent layer 6 and a second absorbent layer 7. The absorbent core 5 has a front portion 5a, a crotch portion 5b and a rear portion 5c. The absorbent core 5 has a core front transverse edge 5a′ and a core rear transverse edge 5c′. The first absorbent layer 6 and the second absorbent layer 7 overlap each other in the crotch portion 5b of the absorbent core 5. The absorbent core 5 additionally comprises a third absorbent layer 8 extending over the crotch portion 5b of the absorbent core 5. The third absorbent layer 8 has a width W_{3c} being smaller than a respective width W_{1c}, W_{2c} of the first and second absorbent layer 6,7 in a respective first and second layer crotch part 6b,7b. The width of the third absorbent layer 8 is smaller along the entire length of the third absorbent layer 8 than the widths of the first and second absorbent layers 6,7. Alternatively, he third absorbent layer 8 may have a width W_{3c} being greater than a respective width W_{1c}, W_{2c} of the first and second absorbent layer 6,7 in a respective first and second layer crotch part 6b,7b.

The first absorbent layer 6 has a first layer frontmost half 6a and a first layer rearmost half 6c, the first absorbent layer 6 furthermore has a first layer front transverse edge 6a′, and a first layer rear transverse edge 6c′. The second absorbent layer 7 has a second layer rearmost half 7c and a second layer frontmost half 7a, the second absorbent layer 7 furthermore has a second layer rear transverse edge 7c′, and a second layer front transverse edge 7a′. The first layer front transverse edge 6a′ corresponds to the core front transverse edge 5a′ and the second layer rear transverse edge 7c′ corresponds to the core rear transverse edge 5c′.

Each of the first absorbent layer 6 and the second absorbent layer 7 are asymmetrical as seen over a transverse centerline T_{c} of the absorbent assembly 2. A maximum width W_{1f} of the first layer frontmost half 6a is greater than a maximum width W₁ᵣ of the first layer rearmost half 6c and a maximum width W₂ᵣ of the second layer rearmost half 7c is greater than a maximum width W₂ᵣ of the second layer frontmost half 7a. The maximum width W_{1f} of the first layer frontmost half 6a may be at least 15% greater, or at least 20% greater than the maximum width W₁ᵣ of the first layer rearmost half 6c. The maximum width W₁ᵣ of the first layer frontmost half 6a may be from 15% to 350% greater, or from 20% to 200% greater, than the maximum width W₁ᵣ of the first layer rearmost half 6c. The maximum width W₂ᵣ of the second layer rearmost half 7c may be from 15% to 350% greater, or from 20% to 200% greater, than the maximum width W₂ᵣ of the second layer frontmost half 7a.

As shown in FIG. 3, the maximum width W₁ᵣ of the first layer rearmost half 6c is greater than a width w_{1c} of a first layer crotch part 6b, the width w_{1c} of a first layer crotch part 6b being measured at a smallest width of the absorbent core 5 where the first absorbent layer 6 and the second absorbent layer 7 overlap each other in the crotch portion 5b. The maximum width W_{2f} of the second layer frontmost half 7a is greater than a width w_{2c} of the first layer crotch part 7b, the width w_{2c} of a first layer crotch part 7b being measured at a smallest width of the absorbent core 5 where the first absorbent layer 6 and the second absorbent layer 7 overlap each other in the crotch portion 5b.

The first absorbent layer 6 has a length 11, as measured along a longitudinal centerline L_{c} of the absorbent undergarment 1, corresponding to from 35% to 95% of a total length l_{ac} of the absorbent core 5, between the front transverse edge 5a′ of the absorbent core 5 and the rear transverse edge 5c′ of the absorbent core 5.

The second absorbent layer 7 has a length 12, as measured along the longitudinal centerline L_{c} of the absorbent undergarment 1, corresponding to from 35% to 95% of the total length l_{ac} of the absorbent core 5, between the front transverse edge 5a′ of the absorbent core 5 and the rear transverse edge 5c′ of the absorbent core.

The first layer front transverse edge 6a′ has a radius of curvature and the second layer front transverse edge 7a′ has a radius of curvature of from ±70% of the radius of curvature of the first layer front transverse edge 6a′. However, alternatively at least a section of the first layer front transverse edge 6a′ may have a radius of curvature and the second layer front transverse edge 7a′ may have a section having radius of curvature of from ±70% of the radius of curvature of the section of the first layer front transverse edge 6a′.

1. A washable and reusable absorbent undergarment comprising an integrated absorbent assembly, the absorbent undergarment having an extension in the longitudinal direction, the absorbent assembly comprising an absorbent core, the absorbent core comprising a first absorbent layer and a second absorbent layer, the absorbent core having a front portion, a crotch portion and a rear portion, the absorbent core having a core front transverse edge and a core rear transverse edge, wherein the first absorbent layer and the second absorbent layer overlap each other in the crotch portions of the absorbent core, the first absorbent layer has a first layer frontmost half and a first layer rearmost half, the first absorbent layer having a first layer front transverse edge, and a first layer rear transverse edge, the second absorbent layer has a second layer rearmost half and a second layer frontmost half, the second absorbent layer has a second layer rear transverse edge, and a second layer front transverse edge, wherein a maximum width of the first layer frontmost half is greater than a maximum width of the first layer rearmost half and wherein a maximum width of the second layer rearmost half is greater than a maximum width of the second layer frontmost half.

2. The washable and reusable absorbent undergarment according to claim 1, wherein the maximum width of the first layer frontmost half is at least 5% greater than the maximum width of the first layer rearmost half.

3. The washable and reusable absorbent undergarment according to claim 1, wherein the maximum width of the second layer rearmost half is at least 5% greater than the maximum width of the second layer frontmost half.

4. The washable and reusable absorbent undergarment according to claim 1, wherein the maximum width of the first layer rearmost half is within the range of from −80% to +20% of a width of a first layer crotch part, the width of the first layer crotch part being measured at a smallest width of the absorbent core where the first absorbent layer and the second absorbent layer overlap each other in the crotch portion.

5. The washable and reusable absorbent undergarment according to claim 1, wherein the maximum width of the second layer frontmost half is within the range of from −80% to +20% of a width of the second layer crotch part, the width of a second layer crotch part being measured at a smallest width of the absorbent core where the first absorbent layer and the second absorbent layer overlap each other in the crotch portion.

6. The washable and reusable absorbent undergarment according to claim 1 wherein a width of the first absorbent layer of the second absorbent layer along an area of the absorbent core where the first absorbent layer and the second absorbent layer overlap each other in the crotch portion.

7. The washable and reusable absorbent undergarment according to claim 1, wherein the first absorbent layer has a length, as measured along a longitudinal centerline of the absorbent undergarment, corresponding to from 35% to 100% of a total length of the absorbent core, between the front transverse edge of the absorbent core and the rear transverse edge of the absorbent core.

8. The washable and reusable absorbent undergarment according to claim 1, wherein the second absorbent layer has a length, as measured along a longitudinal centerline of the absorbent undergarment, corresponding to from 35% to 100%, such as within the range of from 35% to 95%, of a total length of the absorbent core, between the front transverse edge of the absorbent core and the rear transverse edge of the absorbent core.

9. The washable and reusable absorbent undergarment according to claim 1, wherein at least a section of the second layer rear transverse edge has a radius of curvature and wherein at least a section of a first layer rear transverse edge has a radius of curvature of from ±70% of the radius of curvature of the at least a section of the second layer rear transverse edge.

10. The washable and reusable absorbent undergarment according to claim 1, wherein at least a section of the first layer front transverse edge has a radius of curvature and wherein at least a section of a second layer front transverse edge has a radius of curvature of from ±70% of the radius of curvature of the at least a section of the first layer front transverse edge.

11. The washable and reusable absorbent undergarment according to claim 1, wherein the absorbent core comprises a third absorbent layer, the third absorbent layer extending over the crotch portion of the absorbent core.

12. The washable and reusable absorbent undergarment according to claim 11, wherein the third absorbent layer has a width being either smaller or greater than a respective width of the first and second absorbent layer in a respective first and second layer crotch part.

13. The washable and reusable absorbent undergarment according to claim 1, wherein a minimum width of the first layer rearmost fourth is ±80% of a width of a first layer crotch part, the width of the first layer crotch part being measured at a smallest width of the absorbent core where the first absorbent layer and the second absorbent layer overlap each other in the crotch portion.

14. The washable and reusable absorbent undergarment according to claim 1, wherein a minimum width of the second layer frontmost fourth is ±80% of a width of the second layer crotch part, the width of the second layer crotch part being measured at a smallest width of the absorbent core where the first absorbent layer and the second absorbent layer overlap each other in the crotch portion.

15. The washable and reusable absorbent undergarment according to claim 1, wherein the first absorbent layer and the second absorbent layer together form a dog-bone shaped absorbent core.

16. The washable and reusable absorbent undergarment according to claim 1, wherein the absorbent assembly comprises a body-facing layer having a basis weight of from 100 gsm.

17. The washable and reusable absorbent undergarment according to any GAG s claim 1, wherein the absorbent assembly comprises one or more of the following; a woven material, a nonwoven and/or a knit material.

18. The washable and reusable absorbent undergarment claim 1, wherein the absorbent assembly according to comprises a backsheet barrier layer.

19. The washable and reusable absorbent undergarment according to claim 18, wherein the backsheet barrier layer comprises a plastic film.

20. A washable and reusable absorbent pad comprising an absorbent assembly comprising a body-facing layer, a backsheet barrier layer and an absorbent core interposed between the body-facing layer and the backsheet barrier layer, the absorbent core comprising a first absorbent layer and a second absorbent layer, the absorbent core having a front portion, a crotch portion and a rear portion, the absorbent core having a core front transverse edge and a core rear transverse edge, wherein the first absorbent layer and the second absorbent layer overlap each other in the crotch portion of the absorbent core, the first absorbent layer has a first layer frontmost half and a first layer rearmost half, the first absorbent layer having a first layer front transverse edge, and a first layer rear transverse edge the second absorbent layer has a second layer rearmost half and a second layer frontmost half, the second absorbent layer has a second layer rear transverse edge, and a second layer front transverse edge, wherein a maximum width of the first layer frontmost half is greater than a maximum width of the first layer rearmost half and wherein a maximum width of the second layer rearmost half is greater than a maximum width of the second layer frontmost half.

21. The washable and reusable absorbent pad, wherein the absorbent assembly is an absorbent assembly according to claim 2.
